(19) United States
(12) Patent Application Publication
(10) Pub. No.: US 2003/0052009 A1
(43) Pub. Date: Mar. 20, 2003

(54) Method and apparatus for the bulk coating of components

(76) Inventors: Leo L. Case, Rochester Hills, MI (US); Michael R. Morin, Rochester Hills, MI (US); James G. Weslosky, Troy, MI (US); Michael A. Weslosky, Troy, MI (US)

Correspondence Address: POWELL, GOLDSTEIN, FRAZER & MURPHY LLP, P.O. BOX 97233, WASHINGTON, DC, 20090-7223, US

(21) Appl. No.: 09/952,132

(22) Filed: Sep. 14, 2001

(51) Int. Cl. C25D 17/00; G01L 1/20; C02F 1/469; C07K 1/26; G01F 1/64

(52) U.S. Cl. 204/484; 204/198

(57) ABSTRACT

A method and apparatus for the electrocoating of relatively small components is disclosed. The apparatus includes a coating process unit, which is comprised of three component parts, which are defined as the process tank, the transfer system, and the electrode system. Each of the three component parts is integrated with a support structure. The process tank is a fluid-tight, open-top receptacle that holds the coating solution through which the parts are passed. The transfer system generally includes a conveyor chain and transfer media connected with the conveyor chain. The conveyor chain may be electrically insulated. The transfer media connects to the conveyor chain to form the continuous moving support surface onto which the parts are loaded, coated and are unloaded. The transfer media includes a support surface and electrical contact. The electrode system includes both an electrode and an electrode contact of opposite polarity.

### TECHNICAL FIELD

The present invention relates generally to the coating of components. More particularly, the present invention relates to a method and apparatus for the bulk electrocoating of relatively small components. The method generally includes: the preparation of the components to be coated, the placement of the components on transfer media in the form of a movable conveyor, the movement of the transfer media and its accompanying components through a coating-filled process tank, the subjecting of the uncoated parts to an electrically charged coating solution where in the parts are coated, and the conveyed removal of the coated parts to a curing/post treatment/polymerization means.

### SUMMARY OF RELATED ART

Electrocoating or electrodeposition is a process undertaken by the use of opposite polarity electrodes disposed in a spaced-apart array in a tank having a coating bath. The coating process is actually the application of coating by electrolysis and the charged particles, in this case the paint or coating solids, are attracted to the substrate forming an even and uniform deposit of coating thereupon.

Because conductivity is a requirement for electrocoating, the coated article must be conductive or at least must bear an electrically conductive surface. Accordingly, articles to be coated include those manufactured from steel, iron, zinc die-cast, aluminum, copper, brass, or composite materials. This type of coating is particularly valuable where the parts are of complex shapes having recesses and other difficult-to-coat surfaces. This method of coating is also valuable where many like parts must be coated with an identical coating on a volume basis. Accordingly, electrocoating has particular utility in the coating of appliances, metal furniture and in the automotive industry.

Many examples of the electrocoating processes are known in the industry. Some of these include rack and drum coating, the latter being represented by U.S. Pat. No. 5,433,834, issued to Beiz et al. on Jul. 18, 1995. Another popular arrangement for electrocoating is characterized in the suspension or dipping system, whereby a plurality of parts are hung from an overhead conveyor and are pulled through an electrocoating bath. Examples of this approach include U.S. Pat. No. 4,263,122, issued to Urquhart on Apr. 21, 1981, U.S. Pat. No. 4,668,358, issued to Ball on May 26, 1987, U.S. Pat. No. 4,844,783, issued to Takahashi et al. on Jul. 4, 1989, and U.S. Pat. No. 6,139,708, issued to Nonomura et al. on Oct. 31, 2000.

However, attention has been recently directed to the use of conveyor systems upon which are loaded parts. The parts are then carried through and out of an electrocoating tank. An example of this approach includes U.S. Pat. No. 5,810,987, issued to Opitz on Sep. 22, 1998.

While the above-noted approaches represent improvements in the state of the art, there still remains room for improved electrocoating techniques.

### SUMMARY OF THE PRESENT INVENTION

The present invention discloses a method and apparatus for the electrocoating of relatively small components. The apparatus may be generally defined as a coating process unit. The coating process unit is comprised of three component parts, which are defined as the process tank, the transfer system, and the electrode system. Each of the three component parts is integrated with a support structure. The process tank is a fluid-tight, open-top receptacle that holds the coating solution through which the parts are passed. The transfer system generally includes a conveyor chain and transfer media connected to the conveyor chain. The conveyor chain may be electrically insulated. The transfer media connects to the conveyor chain to form the continuous moving support surface on which the parts are loaded, coated and unloaded. The transfer media includes a support surface and electrical contact. The electrode system includes both an electrode and an electrode contact of opposite polarity.

The method of the invention generally includes the preparation of the parts to be coated (by cleaning and, preferably, by phosphating), the introduction of the components onto the transfer media (the conveyor), the movement of the transfer media and its accompanying parts to be coated through the coating-filled process tank, the subjecting of the prepared parts to an electrically charged coating solution while in the process tank, and the conveyed removal of the parts to a post treatment and/or curing area as required. During the coating operation, the conductive portions of the transfer media are coated simultaneously with the parts. Accordingly, following the unloading of the coated parts, the top surface of the transfer media is cleaned to remove the deposited but uncured coating. This step helps to provide positive electrical contact of the parts and, thus, optimal coating results.

The present invention finds broad utility in a variety of painting and coating applications, particularly where small parts are presently being manually fixtured for coating. For example, the present invention would be desirable in painting small parts, i.e. fasteners, clamps, brackets, etc. The coating apparatus of the present invention finds particular application in the painting of small parts, which require a uniform film of highly corrosion resistant coating applied over irregularly-shaped items.

### BRIEF DESCRIPTION OF THE DRAWINGS

The present invention will be more fully understood by reference to the following detailed description of the preferred embodiments of the present invention when read in conjunction with the accompanying drawings, in which like reference characters refer to like parts throughout the views, and in which:

FIG. 1 is an elevational side view of the present invention, illustrated in partial shadow lines, and showing the primary components of the bulk electrocoating apparatus;

FIG. 2 is a top plan view of the present invention, illustrated in partial shadow lines; and

FIG. 3 is an elevational end view of the input end of the present invention, illustrated in partial shadow lines; and

### DETAILED DESCRIPTION OF THE PREFERRED EMBODIMENT

The drawings disclose the preferred embodiment of the present invention. While the configurations according to the illustrated embodiment are preferred, it is envisioned that alternate configurations of the present invention may be adopted without deviating from the invention as portrayed. The preferred embodiment is discussed hereafter.

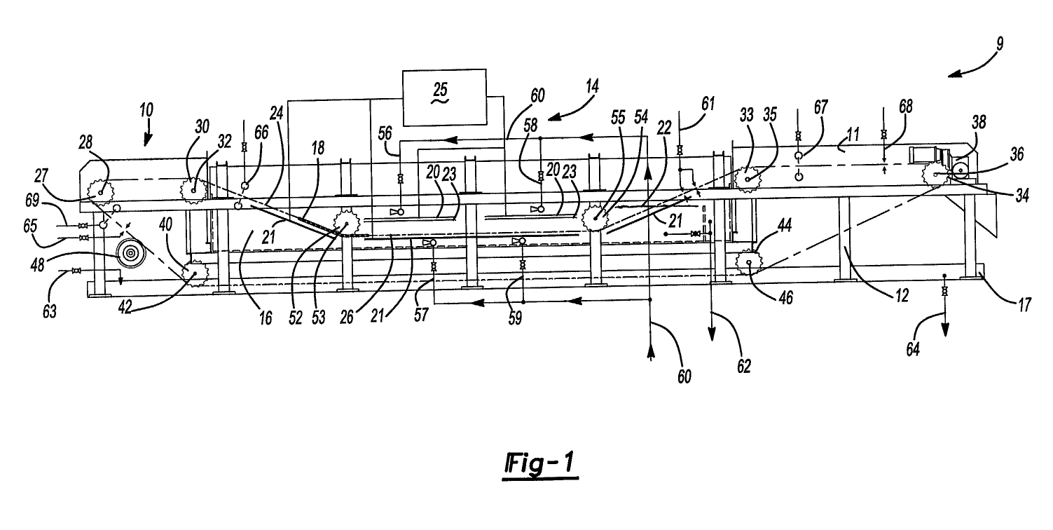

Referring primarily to FIG. 1, a side elevational view of the present coating apparatus, generally illustrated as 9, is shown. The apparatus 9 includes a parts input end 10, an output end 11, a support portion 12 and a coating portion 14. The support portion 12 may be defined by a frame arrangement (as shown) or may be of any other supportive configuration (a cast cement base, for example).

The coating portion 14 includes a coating or process tank 16, a transfer system, generally illustrated as 18, an electrode 20, and an opposite polarity electrode contact 21. The electrode 20, and the electrode contact 21, are supplied D.C. power by a rectifier 25 or other appropriate external D.C. power source.

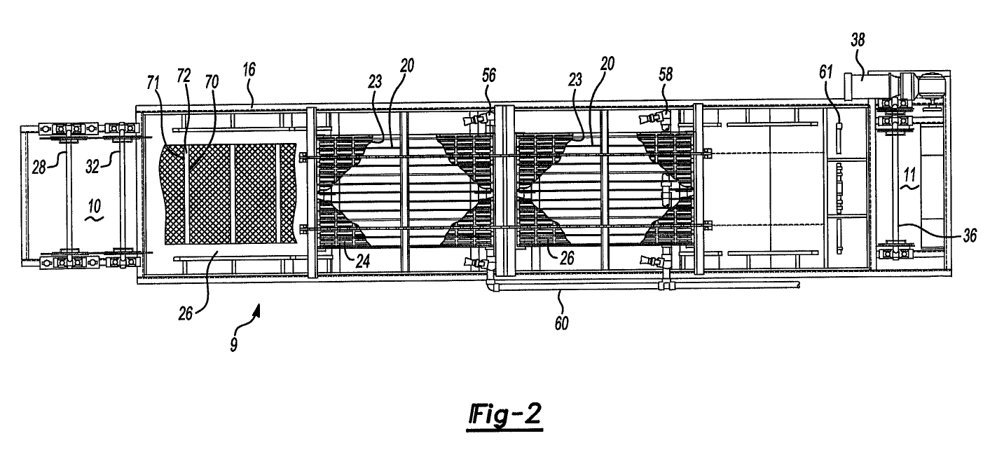

The electrode network composed of the electrodes 20 and 21 forms a plane that is substantially parallel to both the opposing electrode and the transfer media 26. These electrodes 20 and 21 may be a single unit or multiple units. The use of multiple units will allow the use of multiple D.C. sources for multiple voltage potentials or differently sized electrodes for multiple current densities. The electrodes are mounted in an easily removed assembly, which includes an insulating grid 23 below the electrode to prevent accidental short circuits should parts stack excessively on the transfer media 26. This design allows very close proximity of the electrode to the parts which reduces the required voltage and improves the overall coating efficiency. As illustrated in FIG. 2, the electrodes are shown as bare conductive plates, although it is to be understood that other electrode configurations; such as tubular flushable electrodes, may be used.

The process tank 16 is a fluid-tight, open-top receptacle that holds the coating solution through which the parts are passed. The solution level 22 in the process tank must maintained to fully submerge the electrodes and the parts as they pass through the solution.

The electrocoating solution and permeate will be supplied to the process tank 16 from a fluid control module (not shown) of the conventional type which will condition the solutions to maintain the coating bath within the coating manufacturer's specifications. The holding capacity of the fluid control module shall be sufficient to allow complete draining of the process tank 16 for easy maintenance. The preferred arrangement would elevate the process tank sufficiently high to allow gravity drainage to the fluid control module. The fluid control module and its component parts may be configured as needed with respect to holding and transfer capacities.

A watertight pan 17 is provided below the process tank 16 to rinse the coating, by immersion, that may adhere to the conveyor chains 24, 24′ and the transfer media 26. Fresh permeate solution is continuously supplied to this pan from the fluid control module through header 63 and is overflowed back to the fluid control module through header 64. This arrangement facilitates cleaning of the transfer media 26 and conveyor chains 24 and 24′, reduces paint waste and prevents air drying of the coating on the transfer media 26; hence increases the efficiency of the coating process.

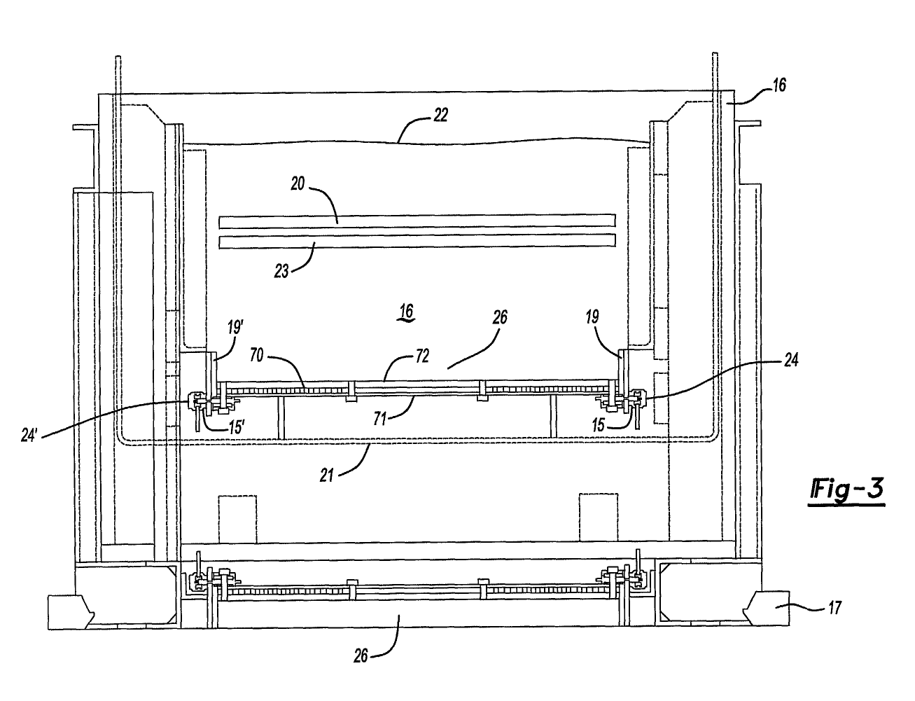

The transfer system 18 includes a pair of spaced apart, parallel conveyor chains 24, 24′ (the latter shown in FIG. 3) and a transfer media 26 suspended by the conveyor chains 24, 24′. (Because the conveyor chains 24, 24′ are identical, and because reference is being made primarily to FIG. 1 wherein only conveyor chain 24 is shown, the present discussion will focus only on the conveyor chain 24. However, it is to be understood that the discussion is to have equal application to the conveyor chain 24′.)

The conveyor chain 24 may be metal or may be composed of a non-conducting material. Alternatively, a metal chain may be modified by having some of its metal links replaced with insulated connectors 15, 15′ at selected intervals along the length of the chain 24. The purpose for this modification is to electrically insulate or to isolate certain segments of the conveyor chain 24 as may be desired. In addition, this arrangement allows for electrical contact to be made at the parts input end 10 of the system, thereby insuring “live entry” of the parts; and reduces the possibility of stray current loops at the load and unload areas.

The conveyor chain 24 is carried on the apparatus 9 by way of a series of sprockets that support, drive, and guide the chain 24. These elements have like counterparts on the opposite (that is, unseen) side of the apparatus 9, and, accordingly, it is to be understood that while only the sprockets on the side shown on the side shown in the view of FIG. 1 will be discussed, each of these elements has a counterpart.

Included in this array is an outer input end upper idler sprocket 27 that is fixed to an outer input end upper idle shaft 28, an inner input end upper idler sprocket 30 that is fixed to an inner input end upper idle shaft 32, an inner output end upper idler sprocket 33 that is fixed to an inner output end upper idle shaft 35, a drive sprocket 34 that is fixed to a drive shaft 36, and a drive 38. The drive 38 can be of any standard industrial type that is known to those skilled in the art for such operations. However, for maximum operational flexibility a variable speed conveyor drive is recommended. In addition, an input end lower idler sprocket 40 is fixed to an input end lower idler sprocket shaft 42, and an output end lower idler sprocket 44 is fixed to an output end lower idler sprocket shaft 46. Of course, a greater or lesser number of sprockets and associated shafts may be used as required for complete operation of the apparatus 9. Sprockets and shafts can be replaced by skids, slides, guides or pulleys, etc. Guidance for the conveyor chain 24 in the coating portion 14, is provided by an input end idler sprocket 52 and shaft 53 and an output end idler sprocket 54 and shaft 55.

As noted above, connected between the conveyor chains 24, 24′ is the transfer media 26 (shown in FIG. 3). The transfer media 26 comprises a plurality of conductive media segments 70 attached to the conductive contact bars 71 provided. The transfer media 26 is secured in place by fastening a mating hold down bar 72 to the contact bar. Each of the conductive segments makes sliding electrical contact with the electrode contact 21 through the contact bars 71. This also provides continuous cleaning of the electrical contact.

The combination of the conveyor chains 24, 24′ and transfer media 26 form a continuous moving support surface onto which the parts to be coated are loaded, coated, and unloaded. Edge guards 19, 19′ may be provided to contain the small parts on the transfer media 26. These edge guards may be affixed to the transfer media 26, the conveyor chains 24, 24′ or the tank 16 walls. By providing the transfer media 26 as a plurality of individual segments, repair and replacement of worn segments may be made individually, thus avoiding the need to replace the entire medium.

To create the desired electrical potential of the transfer media 26, one electrode 20 is situated above the transfer media 26 and the other electrode 21 (opposite polarity) contacts the transfer media 26. Of course, the charge of the electrodes may be reversed and the positioning of the electrodes may be re-arranged, as long as the transfer media 26 is positioned between the electrodes to create the required potential to accomplish electrocoating. Preferably, the transfer media is at ground potential (for safety) regardless of anodic or cathodic operation.

As noted above, a conventional fluid control module provides the conditioned electrocoating solution. The coating solution is conveyed into the apparatus 9 by way of an array of eductors strategically disposed within the tank 16. With particular reference to FIG. 1, the eductors are provided as a first row of coating eductors 56 (upper), 57 (lower) and a second row of coating eductors 58 (upper), 59 (lower) and are fluidly connected by way of a common line 60 which is connected to the fluid control module (not shown). These eductors provide a continuous feed of coating to the tank 16 so as to maintain the minimum required coating liquid level 22. The coating bath solution is overflowed back to the fluid control module through header 62. They also provide continuous circulation of the electrocoating bath, thereby reducing the settling of coating solids, excessive foam generation, or localized temperature variations.

In operation, the parts to be coated must be externally pretreated (not shown) followed by a thorough rinsing in deionized water immediately before the coating process. The cleaned parts are then loaded (manually or automatically) on the input end 10 of the transfer media 26. The parts may either be loaded while the transfer media 26 is moving or may be loaded while the media 26 is stationary. In either event, the parts are transported by the transfer media 26 through an optional dual air knife 66 to remove excess deionized water and then through the electrocoating bath and eventually resurface at the output end 11 of the apparatus 9. After the parts resurface; the coated articles, the returning portion of the conveyor chain 24, and the transfer media 26 pass through a dual permeate spray from header 61 (supplied by the fluid control module), an air knife 67 to remove any excess liquid, and an optional deionized water spray from header 68. The coated parts are then off-loaded for post treatment and/or curing.

The apparatus 9 also provides a method of maintaining optimal coating of the parts by utilizing a method for cleaning the conductive portions of the transfer media 26. Following the unloading of the coated parts, the top surface of the transfer media is cleaned by a rotating brush 48 or alternative method to remove the deposited, but uncured coating. This may be done in conjunction with a permeate spray 65 and an air knife 69. By maintaining the transfer media in a properly cleaned condition, electrical contact between the individual parts and transfer media 26 is improved; thereby assuring consistent application.

A timer or trip mechanism [neither shown] may be provided to automatically rinse the conveyor chain 24 and the transfer media 26 for a timed system shutdown.

Those skilled in the art can now understand from the foregoing description that the broad aspects of the present invention can be implemented in a variety of forms. Therefore, while this invention has been described in connection with particular examples thereof, the true scope of the invention should not be so limited since other modifications will become apparent to the skilled practitioner upon a study of the drawings, specification and the following claims.

What is claimed is:

1. A method for the bulk painting of components includes the steps of:
Forming a bulk coating paint process unit including a substantially fluid-tight process tank for holding paint, a transfer system comprising a conveyor having replaceable, segmented media for conveying the components through the process tank, and electrodes and a support structure for substantially supporting said tank, system and electrodes;
placing a quantity of conductive paint into said process tank;
electrically charging said conductive paint with said electrodes;
surface treating a selected plurality of unpainted components to be painted, said unpainted components being selected from the group consisting of an electrically conductive component or a component having an electrically conductive surface;
placing said selected plurality of unpainted components on said segmented conveyor;
conducting said conveyor with said carried unpainted components into said process tank and through said quantity of conductive paint;
subjecting said unpainted components to said electrically-charged conductive paint while in said process tank whereby said conductive paint is coated on said components through a process of electrodeposition;
conducting said conveyor with the carried painted components out of said process tank;
unloading said now-painted components from said conveyor; and
brushing at least a portion of the top surface of said segmented conveyor to remove residual uncured paint.

2. The method for the bulk painting of components according to claim 1, wherein said electropainting of said components is done in a substantially continuous manner.

3. The method for the bulk painting of components according to claim 1, wherein said placement of said quantity of components on said segmented conveyor may be done in single or plural layers.

4. The method of the bulk painting of components according to claim 1, wherein the segmenting of said segmented conveyor allows said components to be placed in lots on each of said segments for lot grouping.

5. The method of the bulk painting of components according to claim 1, wherein said electrodes include an upper set of electrodes and a lower set of electrodes, each set of upper and lower electrodes being represented by a plurality of individual adjacent sections, whereby each section of said plurality of individual sections may be given a charge that is different from an adjacent section for adjusting the paint coating.

6. The method of the bulk painting of components according to claim 1, wherein said bulk coating paint process unit further includes a spray assembly for spraying a cleaning solution on said segmented conveyor in a substantially continuous manner for improving electrical conductivity between said segmented conveyor and said components.

7. The method of the bulk painting of components according to claim 1, wherein said transfer system further includes a conveyor chain assembly, said segmented conveyor being attached to said conveyor chain assembly.

8. The method of the bulk painting of components according to claim 7, wherein said bulk coating paint process unit further includes a pan having a quantity of permeate provided therein, said segmented conveyor and said conveyor chain assembly being passed through said permeate in said pan for cleaning.

9. The method of the bulk painting of components according to claim 7, wherein said bulk coating paint process unit further includes a plurality of conveyor isolating connectors connecting said conveyor chain assembly and said segmented conveyor for isolating said conveyor chain assembly from said segmented conveyor whereby the material from which said conveyor segments is composed may be broadly varied.

10. The method of the bulk painting of components according to claim 7, wherein said conveyor chain assembly forms the structural foundation to which said conveyor segments are attached, thereby directing the drive forces to and through the conveyor chain assembly, thus relieving tension from said conveyor segments.

11. The method of the bulk painting of components according to claim 1, wherein electrical contact between said segmented conveyor and at least some of said electrodes is made while said segmented conveyor is submerged in said conductive paint.

12. The method of the bulk painting of components according to claim 1, wherein said bulk coating paint process unit may be either an anodic or a cathodic arrangement.

13. The method of the bulk painting of components according to claim 1, wherein transfer system comprises a plurality of side-by-side transfer systems that share said fluid-tight process tank.

14. The method of the bulk painting of components according to claim 1, wherein said transfer system may comprises a plurality of substantially independent transfer system segments that are aligned to operate as a single conveyor system.

15. An apparatus for the continuous bulk painting of components being either composed of a conductive material or having a conductive surface, the apparatus comprising:
a bulk coating paint process unit, said unit including a substantially fluid-tight process tank for holding paint, a component transfer system operatively related to said bulk coating paint process unit, electrodes operatively related to said bulk coating paint process unit, and a support structure for substantially supporting said tank, said transfer system, and said electrodes, said transfer system including a drive system and a plurality of removable and interchangeable segmented media operatively associated with said drive system.

16. The apparatus for the continuous bulk painting of components of claim 15, wherein said drive system comprises a pair of parallel chain drives between which said interchangeable segmented media are suspended.

17. The apparatus for the continuous bulk painting of components of claim 16, further including insulating elements disposed between said chain drives and said interchangeable segmented media for providing a supportive connection therebetween.

18. The apparatus for the continuous bulk painting of components of claim 14, including a paint-removing pan for supplying a paint-removing fluid and a paint-removing fluid delivery system for removing paint from said drive system and said segmented media, said paint-removing pan being positioned generally below said process tank.

19. The apparatus for the continuous bulk painting of components of claim 18, wherein said paint-removing delivery system comprises at least one spray nozzle and a fluid line connecting said paint-removing pan.

20. The apparatus for the continuous bulk painting of components of claim 14, wherein said transfer system has an outer side and an inner side and wherein said electrodes include an upper set of electrodes and a lower set of electrodes, said upper set of electrodes being positioned substantially adjacent said outer side of said transfer system and said lower set of electrodes being positioned substantially adjacent said inner side of said transfer system.

21. The apparatus for the continuous bulk painting of components of claim 20, wherein each set of said upper and said lower electrodes are embodied in a plurality of individual adjacent sections.

22. The apparatus for the continuous bulk painting of components of claim 14, wherein said transfer system comprises a plurality of side-by-side transfer systems that share said fluid-tight process tank.

23. The apparatus for the continuous bulk painting of components of claim 14, wherein said transfer system may comprises a plurality of substantially independent transfer system segments that are aligned to operate as a single conveyor system.

24. An apparatus for the continuous bulk painting of components being either composed of a conductive material or having a conductive surface, the apparatus comprising:
a bulk coating paint process unit, said unit including a substantially fluid-tight process tank for holding paint, a component transfer system operatively related to said bulk coating paint process unit, electrodes operatively related to said bulk coating paint process unit, and a support structure for substantially supporting said tank, said transfer system, and said electrodes, said transfer system including a drive system and a plurality of removable and interchangeable segmented media operatively associated with said drive system, the apparatus further including a paint-removing pan for supplying a paint-removing fluid and a paint-removing fluid delivery system for removing paint from said drive system and said segmented media, said paint-removing pan being positioned generally below said process tank.
